(19) United States
(12) Patent Application Publication
(10) Pub. No.: US 2002/0123643 A1
(43) Pub. Date: Sep. 5, 2002

(54) Process for producing methylcyclohexyl (METH) acrylates

(75) Inventor: Jean-Micheal Paul, Metz (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: ATOFINA, Puteaux (FR)

(21) Appl. No.: 09/983,768

(22) Filed: Oct. 25, 2001

(30) Foreign Application Priority Data
Oct. 25, 2000 (FR) 0013672

(51) Int. Cl. C07C 57/04; C07C 51/09; C07C 67/03

(52) U.S. Cl. 560/217

(57) ABSTRACT

A methylcyclohexyl (meth)acrylate with formula (I) is prepared by reacting a lower (meth)acrylate (II) with an alcohol (III) in the presence of a transesterification catalyst selected from titanium, tin, zirconium, magnesium, calcium, lithium, potassium or sodium alcoholates; chelates of zirconium, calcium, magnesium or lithium with 1,3-dicarbonyl compounds; dialkyltin oxides, dialkyltin dialkoxides, dialkyltin diesters and distannoxanes and magnesium, calcium or lithium hydroxides.

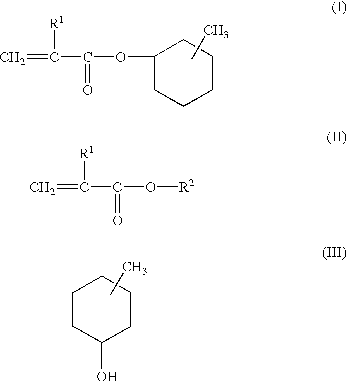

R¹=CH₃; the CH₃ group substituting a cyclohexyl radical in (I) and (III) being able to occupy any of the ortho, meta or para positions; and R²=C₁-C₄ alkyl.

The present invention relates to a process for producing methylcyclohexyl (meth)acrylates, more precisely 2-methylcyclohexyl, 3-methylcyclohexyl and 4-methylcyclohexyl (meth)acrylates, individually or as a mixture of at least two thereof.

Such monomers endow resins formed from compositions containing them with particular properties, in particular good heat resistance, an improvement in certain mechanical properties, and an improvement in the appearance of coatings.

Different synthesis processes have been described in the literature:

esterification of acrylic acid with 4-methylcyclohexanol in the presence of para-toluenesulphonic acid (Azerb. Khim. Zh. 1983, 357-9; J. Polymer Sci., 1965, 3(11)3978-81); and

transesterification of methyl acrylate with 2-, 3- and 4-methylcyclohexanols in the presence of para-toluenesulphonic acid (U.S. Pat. Nos. 2,445,925; 2,473,544).

In its search to produce such methylcyclohexyl (meth)acrylates with better yields and selectivities, the Applicant has discovered a novel family of transesterification catalysts that can achieve the required aims, namely excellent yields and very good selectivity.

Thus, the present invention provides a process for producing a methylcyclohexyl (meth)acrylate with formula (I):

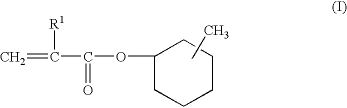

where:

R¹ represents H or CH₃;

the CH₃ group substituting the cyclohexyl radical can occupy any of the positions ortho, meta or para to the (meth)acryloyloxy group;

in which a lower (meth)acrylate with formula (II):

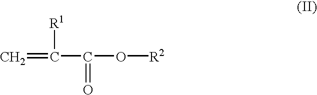

where:

R¹ is as defined above; and

R² represents a C₁-C₄ alkyl radical is reacted in the presence of a transesterification catalyst with at least one alcohol with formula (III):

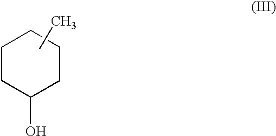

where the CH₃ group substituting the cyclohexyl radical can occupy any of the positions ortho, meta or para to the OH group,

characterized in that the transesterification catalyst employed is a compound selected from

titanium, tin, zirconium, magnesium, calcium, lithium, potassium or sodium alcoholates

chelates of zirconium, calcium, magnesium or lithium with 1,3-dicarbonyl compounds;

dialkyltin oxides, dialkyltin dialkoxides, dialkyltin diesters and distannoxanes; and

magnesium, calcium or lithium hydroxides.

Alcohol (III) can be used in the form of its pure cis or trans isomers, or as cis/trans mixtures.

Mixtures of positional isomers of compound (I) are obtained when mixtures of positional isomers of alcohol (III) are used.

Examples of alcoholates are tetraalkyl titanates Ti(OR¹)₄ where R¹ represents methyl, ethyl, butyl, isopropyl, 2-ethylhexyl; and magnesium alcoholates Mg(OR²)₂, R² representing a C₁-C₄ alkyl residue, for example methyl, ethyl, n-propyl, butyl.

Examples of chelates are zirconium, calcium, magnesium and lithium acetylacetonates.

Dialkyltin oxides are in particular compounds with formula R³₂SnO, where R³ represents a C₁-C₃₀ alkyl residue, an example being di-n-butyltin oxide Bu₂SnO (DBTO).

Dialkyltin dialkoxides are in particular compounds with formula R⁴₂Sn(OR⁵)₂, in which R⁴ et R⁵ each independently represent a C₁-C₃₀ alkyl residue.

Dialkyltin diesters are in particular compounds with formula

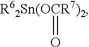

in which R⁶ and R⁷ each independently represent a C₁-C₃₀ alkyl, examples of these compounds being dibutyltin dilaurate and dibutyltin diacetate.

Distannoxanes are in particular compounds with formula XR⁸₂SnOSnR⁸₂Y, in which X and Y each independently represent Cl, Br, NCS or OH; and R⁸ each represent a C₁-C₈ alkyl such as methyl or butyl. An example that can be cited is tetrabutyldichlorodistannoxane.

In accordance with the present invention, a catalytic quantity of catalyst preferably in the range 10⁻³ to 5×10⁻² mol, in particular in the range 5×10⁻³ to 5×10⁻² mol per mol of alcohol with formula (III) is employed.

The lower (meth)acrylate (II) is, for example, methyl, ethyl, n-propyl, isopropyl, n-butyl, isobutyl or tert-butyl (meth)acrylate, in particular methyl (meth)acrylate.

The reaction of the process of the present invention can be carried out in the presence of an excess of one or other of the reactants. However, it is preferably carried out in the presence of an excess of lower ester (II).

The lower ester (II)/alcohol (III) molar ratio can therefore, in general, be in the range 0.7 to 7, preferably in the range 2 to 4.

Moreover, the reaction of the process of the invention is preferably carried out in the presence of at least one polymerization inhibitor, the latter being selected in particular from phenothiazine, copper butyldithiocarbamate, hydroquinone monomethyl ether, hydroquinone, di-tert-butyl-para-cresol, 2,2,6,6-tetramethyl-1-piperidyloxy (TEMPO), 4-hydroxy-2,2,6,6-tetramethyl-1-piperidyloxy (4-hydroxy-TEMPO), 4-methoxy-2,2,6,6-tetramethyl-1-piperidyloxy (4-methoxy-TEMPO), 4-oxo-2,2,6,6-tetramethyl-1-piperidyloxy (4-oxo-TEMPO), and mixtures thereof in any proportions.

In particular, the polymerization inhibitor or inhibitors is/are introduced in effective quantities, preferably in an amount of 0.05% to 0/5% by weight with respect to the alcohol with formula (III).

The reaction of the process of the invention is preferably carried out under reduced pressure to keep the temperature of the reaction mixture below 120° C.

The reaction period depends on the operating conditions, but is generally in the range 5 to 8 hours.

In a particular implementation of the process according to the present invention, the reaction is carried out using the following steps in succession:

mixing the lower (meth)acrylate with formula (II), alcohol or alcohols (III) and polymerization inhibitor or inhibitors, if required, and heating under reflux to eliminate residual traces of moisture in the form of a lower (meth)acrylate (II)/water azeotrope;

when the drying step is complete, introducing the catalyst and initiating the reaction phase, the lower alcohol that forms during the reaction being eliminated in the form of a lower (meth)acrylate (II)/lower alcohol azeotrope;

purifying, by purification, the crude reaction mixture by eliminating the residual lower ester (II) and residual alcohol (III) in the form of two distillation fractions; and recovering the desired compound (I) or a mixture of desired compounds (I) in the form of a third distillation fraction.

The following examples illustrate the present invention without in any way limiting its scope. In these examples, the percentages are percentages by weight unless otherwise indicated and the following abbreviations have been used

MAM: methyl methacrylate

Zr(acac)₂: zirconium acetylacetonate

DBTO: di-n-butyltin oxide, Bu₂SnO

### EXAMPLE 1

The following were introduced in succession into a 1 liter glass reactor stirrred with an anchor-type agitator, heated with a double-walled jacket supplied with thermostatted oil and surmounted by a packed distillation column with an efficiency of 4 theoretical plates:

251.2 g of 3-methylcyclohexanol

660 g of MAM;

0.80 g of phenothiazine; and

0.80 g of copper dibutyl dithiocarbamate.

The following various operations were carried out in succession:

Drying:

Heating under reflux at atmospheric pressure, distilling a head containing 0.05% water (fraction F1: 50 g).

Throughout the test period, air was bubbled into the reaction mixture.

Reacting:

When drying was complete, the DBTO catalyst was introduced (8.22 g, i.e., 0.015 mol/mol of 3-methyl-cyclohexanol). The pressure was gradually reduced to 6.66×10⁴ Pa (500 mmHg), to keep the temperature in the reactor below 100° C.

125 g of a mixture consisting of 56.9% of methanol and 42.7% of methyl methacrylate was recovered as overhead (fraction F1).

The reaction period was 6.5 h.

The degree of conversion, calculated from the methanol formed, was >99%.

Distilling:

The excess MAM and residual traces of alcohol (III) were eliminated by distillation, by gradually reducing the pressure from 6.66×10⁴ Pa (500 mmHg) to 3.99×10³ Pa (30 mmHg) to keep the temperature in the reactor to a maximum of 115° C. (fraction F2: 365 g).

The 3-methylcyclohexyl methacrylate was distilled under 2.66×10³ Pa (20 mmHg) (fraction F3 348 g).

The 3-methylcyclohexyl methacrylate finally obtained was a mixture of cis and trans isomers. It was characterized by NMR.

### EXAMPLES 2 TO 7

Six compounds or mixtures of compounds of the invention were prepared under the same general conditions as those employed in Example 1, varying the MAM/alcohol (III) molar ratio, the nature of alcohol (III), the nature of the catalyst and the amount of the latter.

The results are shown in Table 1.

TABLE 1

| | | | | Degree of | |
|---|---|---|---|---|---|
| | MAM/ | | | conversion | |
| | Alcohol | | | of alcohol | |
| | (III) | | | (III) | Selectivity |
| Examples | molar ratio | Alcohol (III) | Catalyst | (%) | (%) |
| 2 | 3 | 4-methylcyclohexanol | Zr(acac)₂ | >99 | >98 |
| | | | 0.015 mol/mol alcohol (III) | | |
| 3 | 3 | 3-methylcyclohexanol 69% | DBTO | >99 | >98 |
| | | 4-methylcyclohexanol 31% | 0.015 mol/mol alcohol (III) | | |
| 4 | 2 | 3-methylcyclohexanol 69% | DBTO | 97 | >98 |
| | | 4-methylcyclohexanol 31% | 0.015 mol/mol alcohol (III) | | |
| 5 | 3 | 3-methylcyclohexanol 69% | Zr(acac)₂ | 99 | >98 |
| | | 4-methylcyclohexanol 31% | 0.01 mol/mol alcohol (III) | | |
| 6 | 2.5 | 3-methylcyclohexanol 69% | Tetrabutyldichlorodistannoxane | 98 | >98 |
| | | 4-methylcyclohexanol 31% | 0.01 mol/mol alcohol (III) | | |
| 7 | 3 | 2-methylcyclohexanol | DBTO | >99 | >98 |
| | | | 0.02 mol/mol alcohol (III) | | |

The preceding examples can be repeated with similar success by substituting the generically or specifically described reactants and/or operating conditions of this invention for those used in the preceding examples. Also, the preceding specific embodiments are to be construed as merely illustrative, and not limitative of the remainder of the disclosure in any way whatsoever.

The entire disclosure of all applications, patents and publications, cited above and below, and of corresponding French application 00/13.672, are hereby incorporated by reference.

From the foregoing description, one skilled in the art can easily ascertain the essential characteristics of this invention, and without departing from the spirit and scope thereof, can make various changes and modifications of the invention to adapt it to various usages and conditions.

1. Process for producing a methylcyclohexyl (meth)acrylate with formula (I): 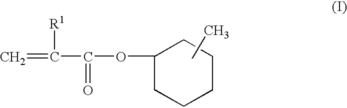
where:
R¹ represents H or CH₃;
the CH₃ group substituting the cyclohexyl radical can occupy any of the positions ortho, meta or para to the (meth)acryloyloxy group:
in which a lower (meth)acrylate with formula (II): 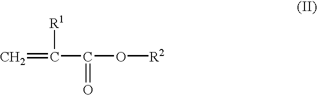
where:
R¹ is as defined above; and
R² represents a C₁-C₄ alkyl radical is reacted in the presence of a transesterification catalyst with at least one alcohol with formula (III): 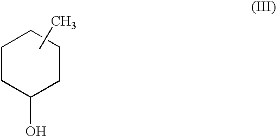
where the CH₃ group substituting the cyclohexyl radical can occupy any of the positions ortho, meta or para to the OH group,
characterized in that the transesterification catalyst employed is a compound selected from: titanium, tin, zirconium, magnesium, calcium, lithium, potassium or sodium alcoholates; chelates of zirconium, calcium, magnesium or lithium with 1,3-dicarbonyl compounds; dialkyltin oxides, dialkyltin dialkoxides, dialkyltin diesters and distannoxanes; and magnesium, calcium and lithium hydroxides.

2. Process according to claim 1, characterized in that the catalyst used is a compound selected from tetraalkyl titanates Ti (OR¹)₄ where R¹ represents methyl, ethyl, butyl, isopropyl, 2-ethylhexyl; magnesium alcoholates Mg(OR²)₂, R² representing a C₁-C₄ alkyl residue, for example methyl, ethyl, n-propyl, butyl; zirconium, calcium, magnesium or lithium acetylacetonates; di-n-butyltin oxide; dibutyltin dilaurate; dibutyltin acetate; and tetrabutyldichlorodistannoxane.

3. Process according to claim 1 or claim 2, characterized in that the quantity of catalyst used is in the range 10⁻³ to 5×10⁻² mols per mol of alcohol with formula (III).

4. Process according to claim 3, characterized in that the quantity of catalyst employed is in the range 5×10⁻³ to 5×10⁻² mols per mol of alcohol with formula (III).

5. Process according to any one of claims 1 to 4, characterized in that the lower (meth)acrylate (II) employed is methyl, ethyl, n-propyl, isopropyl, n-butyl, isobutyl or tert-butyl (meth)acrylate.

6. Process according to any one of claims 1 to 5, characterized in that the reaction is carried out in the presence of an excess of one or other of the reactants.

7. Process according to claim 6, characterized in that the reaction is carried out using a lower ester (II)/alcohol (III) molar ratio in the range 0.7 to 7.

8. Process according to claim 7, characterized in that the reaction is carried out using a lower ester (II)/alcohol (III) molar ratio in the range 2 to 4.

9. Process according to any one of claims 1 to 8, characterized in that the reaction is carried out in the presence of at least one polymerization inhibitor selected in particular from phenothiazine, copper butyldithio-carbamate, hydroquinone monomethyl ether, hydroquinone, di-tert-butyl-para-cresol, 2,2,6,6-tetramethyl-1-piper-idyloxy (TEMPO), 4-hydroxy-2,2,6,6-tetramethyl-1-piperidyloxy (4-hydroxy-TEMPO), 4-methoxy-2,2,6,6-tetramethyl-1-piperidyloxy (4-methoxy-TEMPO), and 4-oxo-2,2,6,6-tetramethyl-1-piperidyloxy (4-oxo-TEMPO).

10. Process according to claim 9, characterized in that the polymerization inhibitor or inhibitors is/are introduced in an amount of 0.05% to 0.5% by weight with respect to the alcohol with formula (III).

11. Process according to any one of claims 1 to 10, characterized in that the reaction is carried out under reduced pressure to keep the temperature of the reaction mixture below 120° C.

12. Process according to any one of claims 1 to 11, characterized in that the reaction is carried out over a time period in the range 5 to 8 hours.

13. Process according to any one of claims 1 to 12, characterized in that the reaction is carried out using the following steps in succession:
mixing the lower (meth)acrylate with formula (II), alcohol or alcohols (III) and polymerization inhibitor or inhibitors, if required, and heating under reflux to eliminate residual traces of moisture in the form of a lower (meth)acrylate (II)/water azeotrope;
when the drying step is complete, introducing the catalyst and initiating the reaction phase, the lower alcohol that forms during the reaction being eliminated in the form of a lower (meth)acrylate (II)/low molecular weight alchohol azeotrope ;
purifying, by distillation, the crude reaction mixture by eliminating the residual lower ester (II) and residual alcohol (III) in the form of two distillation fractions; and recovering the desired compound (I) or a mixture of desired compounds (I) in the form of a third distillation fraction.
